(19) United States
(12) Patent Application Publication
(10) Pub. No.: US 2015/0305690 A1
(43) Pub. Date: Oct. 29, 2015

(54) Medical Monitoring System

(71) Applicant: GEOB INTERNATIONAL SDN. BHD., Bukit Mertajam, Penang (MY)

(72) Inventors: Geoffrey Weng Leng TAN, Kedah (MY); Brandon BEH, Kedah (MY)

(21) Appl. No.: 14/441,496

(22) PCT Filed: Nov. 20, 2013
(86) PCT No.: PCT/MY2013/000200

§ 371 Date: May 7, 2015

(30) Foreign Application Priority Data
Nov. 22, 2012 (MY) PI 2012005042

(51) Int. Cl. A61B 5/00; A61B 5/0205; A61B 5/053; A61B 5/01

(52) U.S. Cl. CPC A61B 5/746; A61B 5/01; A61B 5/4266; A61B 5/02055; A61B 5/0537; A61B 5/0008; A61B 5/0017; A61B 5/6843; A61B 5/742; A61B 5/747; A61B 5/681; A61B 2560/0214; A61B 2560/0475

(57) ABSTRACT

The present invention relates to a medical monitoring system (100) that comprises at least one wearable monitoring device (10) and a wireless transceiver unit (34). The medical monitoring system (100) serving to provision for the monitoring of at least one mobile subject's physiological parameters that include body temperature or perspiration, analyze physiological parameter measurements embodied in the form of electrical signals, produce a medical alert signal in the event the physiological parameter measurements fall beyond predetermined threshold values and further serve to wirelessly transmit the medical alert signal and electrically embodied physiological parameter measurements to a remote monitoring station (32).

The present invention relates broadly to the field of medical monitoring and emergency alert systems. More particularly the present invention relates to a medical monitoring and emergency alert system that provides for wireless transmission of a medical alert to a remote monitoring station to consequently enable remote monitoring and personal emergency response despatch.

### BACKGROUND TO THE INVENTION

A medical alarm system is an alarm system designed to signal presence of a medical hazard requiring urgent attention and to summon a medical emergency personnel. Other terms for medical alarm systems include but are not limited to “Personal Emergency Response System” and “Wireless Medical Emergency Alert Systems”.

A medical monitoring system on the other hand is designed to continuously or periodically transmit electrically embodied physiological parameter measurements embodied as RF signals to a remote monitoring station for physiological monitoring of a subject as well as usually provisioning for alerting of a subject and/or a remote care-giver or remote emergency response team of a medical emergency in relation to the subject's well-being.

These systems, typically find applications in care of the elderly, whose state of health need to be constantly monitored while advantageously still enabling the elderly to retain mobility and consequently maintain an active lifestyle. Moreover, these systems are also being increasingly used across all age categories and even include young children and working adults.

In view of the increasing growth of global ageing population as well as an increase in deaths due to diseases like diabetes and heart attack, it is foreseeable that such medical alerting systems be in great demand. The subsequent passages detail abstracts of prior art documents which relate to prior art medical emergency alert systems as well as drawbacks of the prior art medical emergency alert systems disclosed therein.

U.S. Pat. No. 7,052,472 B1 discloses a system for detecting symptoms of hypoglycaemia in a diabetic individual. U.S. Pat. No. 7,052,472 B1 however does not provide for relaying of a medical emergency alert to a remote care-giver or a remote monitoring station. U.S. Pat. No. 7,052,472 B1 further does not disclose transmission of a medical alert to a remote care-giver or a remote monitoring station by either wire-line or wireless communication channels. The relaying of a medical alert to a remote care-giver or remote monitoring station may prove critical when a wearer is in a situation where he/she is unable to seek medical emergency assistance on his/her own.

US2009/0322513 A1 discloses a medical emergency reporting system and methodology that utilizes a monitoring device to continuously monitor key physiological parameters of a person, and when measurements exceed programmed threshold levels, it automatically issues a medical emergency alert along with location information to a remote monitoring centre via a wireless network and the internet for immediate local response. US2009/0322513 A1 however fails to disclose a alerting system that, provisions for production of an audible alarm to a wearer of the wearable monitoring device in the event the wearable monitoring device is not tightly coupled to the wearer utilizing the measurement obtained by the physiological condition sensor to consequently alert the subject that the wearable monitoring device is improperly worn. US2009/0322513 further fails to disclose a combination of communication channels that comprise of both wire-line and wireless communication channels for relaying of a medical emergency alert to a remote monitoring station or remote care-giver. The provision of wire-line communication may prove to be advantageous as it provides redundancy to wireless communication, in communicating an emergency medical alert to a remote monitoring station. In the event, the wireless channel communication experiences technical problems, an alert may be communicated to the remote monitoring station by way of wire-line communication channels that include the POTS (Plain Old Telephone Service) and PSTN (Public Switched Telephone Network).

U.S. Pat. No. 7,978,063 B2 (refer to FIG. 1) discloses a wireless network for monitoring a patient (10) comprising at least one wearable monitor (12, 70) including a physiological condition sensor (34,74) coupled to the patient (10) to sense and communicate data related to one physiological function of the patient (10). A first body communication unit (16, 78) interfaces with the at least one wearable monitor (12, 70) and communicates with the first body communication unit (16, 78) utilizing the near field capacitive body coupled protocol. An external communication unit (22) communicates the data to a remote medical monitoring station via a cell phone network or the internet. U.S. Pat. No. 7,978,063 similar to US 2009/0322513 A1 does not provide a method of determining whether the wearable monitoring device i.e. wearable monitor is firmly attached to the surface of the skin of a subject being monitored utilizing the measurement obtained by the physiological condition sensor to consequently alert the subject that the wearable monitoring device is improperly worn. Moreover similar to U.S. Pat. No. 7,978,063 B2 does not disclose a system that augments a wireless communication channel for the communication of an alert with either a combination of a wireless communication channel, wire-line communication channel or fibre optic communication channel.

US 2011/0160547 A1 discloses a wireless transmission device for physiological information that is carried personally by a user and is attached on the skin to measure the user's physiological condition by touching the skin. Related physiological signals are continuously and automatically transmitted to other monitoring devices through an automatic wireless transmission unit in the wireless transmission device. Therefore, the user's physical condition can be monitored at any time and the related physiological information can be provided to a medical facility or other monitoring facility for understanding and tracking. US 2011/0160547 A1, similar to prior art documents U.S. Pat. No. 7,052,472 B1, US 2009/0322513 A1 and U.S. Pat. No. 7,978,063 B2, does not disclose the provisioning of a transmission of a medical alert by way of either wire-line or wireless communication channels. In addition US 2011/0160547 A1, does not disclose a wireless transmission device that generates an alert based on measured physiological parameters that fall beyond a predetermine threshold of measurement values, with the threshold values being customizable according to age, gender and medical condition of a particular individual. The provision of customizable threshold values prevents occurrence of false alarms, thus increasing effectiveness of the system for provision of remote monitoring and emergency response initiation.

In view of the above, it is desirable to provide an improved medical monitoring system that overcomes the disadvantages of medical monitoring and emergency alert systems of the prior art.

### SUMMARY OF THE INVENTION

The following presents a simplified summary of the invention in order to provide a basic understanding of some aspects of the invention. This summary is not intended as an extensive overview of the invention. It is not intended to identify key/critical elements of the invention. Its sole purpose is to present some concepts of the invention in a simplified form as a prelude to the more detailed description that is presented later.

In one aspect, the present invention provides an apparatus that, provisions for the remote monitoring and medical emergency alerting of an individual's physiological parameters that includes:
at least one wearable monitoring device that serves to monitor at least one mobile subject's physiological parameters that may include body temperature, perspiration and blood pressure, analyze the physiological parameters, by embodying physiological parameter measurements in the form of electrical signals; produce a medical alert signal in the event the physiological parameter measurements fall beyond predetermined threshold values and further serve to wirelessly transmit the medical alert and electrically embodied physiological parameter measurements by embodying the medical alert and electrically embodied physiological parameter measurements in the form of RF signals; and
a wireless transceiver unit, and that is configured to communicate with the at least one wearable monitoring device via RF wireless communication, consequently enabling receipt of the RF signals embodying the medical alert signal and electrically embodied physiological parameter measurements originating from the at least one wearable monitoring device, processing the received medical alert signal and electrically embodied physiological parameter measurement to provide a local alarm and/or provide a medical alert as well as relay the electrically embodied parameter measurements to a remote monitoring station by way of transmitting the medical alert and electrically embodied parameter measurements over a cellular network or alternatively by way of transmitting the medical alert and electrically embodied physiological parameter measurements via a wire-line communication channel or fiber optic communication channel to the remote monitoring station.

The wireless transceiver unit serving to transmit the medical alert and physiological parameter measurements to a remote monitoring station over a cellular network, a cellular network aggregated to the internet and/or via a PSTN (Public Switched Telephone Network) through the POTS (Plain Old Telephone Service) or a fiber optic communication network to alert a remotely located caregiver or, a remotely, located medical emergency response personnel.

The remote monitoring station in a preferable embodiment includes a cellular phone of a remote care-giver. The remote monitoring station in yet another preferable embodiment includes a remotely located medical institution's call center that is communicably interconnected to the cellular network and/or the PSTN (Public Switched Telephone Network) through the POTS (Plain Old Telephone Service).

The at least one wearable monitoring device of the improved medical monitoring and emergency alert system of the present invention, in a preferable embodiment includes:
at least one physiological sensor that includes at least one body temperature sensor that serves to enable the embodying of at least one physiological parameter measurement such as body temperature measurement and perspiration measurement (measured in terms of % relative humidity) into electrical signals;
a central processing unit that serves to receive the at least one electrically embodied physiological parameter measurement from the at least one physiological sensor and determine if the at least one electrically embodied physiological parameter measurement falls beyond predetermined thresholds; actuate a local alarm in the event a medical alert condition is determined and transmit a medical alert signal and the at least one physiological parameter measurement to a transceiver circuit; and
the transceiver circuit serving to receive the medical alert signal and the at least one electrically embodied physiological parameter measurement transmitted by the central processing unit and subsequently wirelessly transmits the medical alert signal and the at least one physiological parameter measurement to the wireless transceiver unit.

In a preferred embodiment of the medical monitoring system of the present invention, the at least one wearable monitoring device further includes at least one LED (Light Emitting Diode) indicator, a USB (Universal Serial Bus) port for battery charging, a power button and an emergency alert activation button.

In a preferred embodiment of the medical monitoring system of the present invention, the occurrence of a medical alert condition is determined at the wireless transceiver unit. In the preferred embodiment of the medical monitoring system of the present invention, the at least one electrically embodied physiological parameter measurement obtained by the at least one sensor of the wearable monitoring device is received by the wireless transceiver unit, which subsequently determines if the received electrically embodied physiological parameter measurement falls beyond predetermined thresholds.

In a preferred embodiment of the medical monitoring system of the present invention, the wearable monitoring device comprises of at least one physiological sensor that incorporates a body temperature sensing element and a relative humidity sensing element.

In a preferred embodiment of the medical monitoring system of the present invention, the at least one wearable monitoring device takes the form of a dermal patch which comprises of RFID circuitry and at least one sensing element. The at least one sensing element enables the embodying of physiological parameter measurement such as body temperature measurement and measurement of perspiration (i.e. in terms of relative humidity measurement) into electrical signals. The wearable monitoring device in this preferred embodiment, including electronic circuitry that enables transmission of the electrically embodied physiological parameter measurements (by embodying the electrically embodied physiological parameter measurements as RF signals), to the wireless transceiver unit for further processing.

In a preferred embodiment of the medical monitoring system of the present invention, the wireless transceiver unit is a cellular phone.

In a preferred embodiment of the medical monitoring system of the present invention, the at least one wearable monitoring device communicates with the wireless transceiver unit utilizing wireless RF signals in accordance with the IEEE's Bluetooth protocol.

In another preferred embodiment of the medical monitoring system of the present invention, the at least one wearable monitoring device communicates with the wireless transceiver unit utilizing wireless RF signals in accordance with the IEEE's Zig-Bee protocol.

In yet another preferred embodiment of the medical monitoring system of the present invention, the at least one wearable monitoring device communicates with the wireless transceiver unit utilizing wireless RF signals in accordance with the IEEE's 802.11 WLAN protocol.

In yet another preferred embodiment of the medical monitoring system of the present invention, the at least one wearable monitoring device communicates with the wireless transceiver unit by transmitting and receiving wireless RF signals within frequency bands commonly used when RFID technology is deployed.

In one embodiment of the present invention, the medical monitoring system of the present invention provisions for threshold physiological parameter measurement settings that are customizable based on the age, gender, normal medical condition of a subject being monitored and time of the day (to accommodate the amount and type of activities that one undergoes during a particular time of the day).

In one embodiment of the present invention, the medical monitoring system of the present invention provides a wearable monitoring device that incorporates a relative humidity sensor capable of sensing the relative humidity of a subject without the relative humidity sensor being in actual contact with the surface of the skin of the subject.

In another aspect of the present invention, there is provided a method to determine and alert a subject wearing a wearable monitoring device configured to measure a physiological parameter, as to whether, the wearable monitoring device is in firm contact with the subject's skin utilizing an electrically embodied physiological parameter measurement obtained by an at least one sensor of the wearable monitoring device; the method comprising the steps of:
i. acquiring the electrically embodied physiological parameter;
ii. determining if the electrically embodied physiological parameter measurement acquired in step (i) falls beyond a predetermined range of measurements that represent the normal range of measurement values when the wearable monitoring device is tightly coupled to the subject's skin; and
iii. actuating an audible and/or a visual alarm on the wearable monitoring device as well as an audible and/or a visual alert which may include a message on the wireless transceiver unit in the event the electrically embodied physiological parameter measurement exceeds the normal range of measurement values;

In yet another aspect of the medical monitoring system of the present invention there is provided a method to alert a remote care-giver or a remote team of emergency response personnel via a remote monitoring station through the wireless transceiver unit that is in communication with the at least one wearable monitoring device and the remote monitoring station. The method being executed by the wireless transceiver unit and comprises the steps of:
i. acquiring at least one electrically embodied physiological parameter from a wearable monitoring device worn by a subject who is being monitored
ii. determining whether the at least one electrically embodied physiological parameter measurement acquired in step (i) falls beyond a predetermined range of measurements that represent the normal range of physiological parameter measurement values when the subject is healthy;
iii. actuating a local audible and/or visual alarm on the wearable monitoring device and/or the wireless transceiver unit;
iv. automatically executing a phone call to at least one primary number corresponding to at least one remote monitoring station which may include a cell-phone of a remote care-giver or a remote call center of a medical emergency response team; the phone call being executed a predetermined number of times until it is acknowledged by the remote monitoring station;
v. automatically executing a phone call for a predetermined number of times to at least one other alternate number corresponding to at least one remote monitoring station which may include a cell phone of a remote care-giver or a remote call center of a medical emergency response team in the event, the phone call to the predetermined primary number is not acknowledged;
vi. repeating steps (iv) and (v) until either one of the phone calls to the at least one predetermined primary number or the at least one predetermined secondary number is acknowledged; and
vii. automatically executing the wireless transmission of an SMS alert to the at least one predetermined primary number or at least one other number corresponding to at least one remote monitoring station which may include a cell-phone of a remote care-giver.

Additional aspects, features and advantages of the invention will become apparent to those skilled in the art upon consideration of the following detailed description of preferred embodiments of the invention in conjunction with the drawings listed below.

### BRIEF DESCRIPTION OF THE DRAWINGS

The present invention will be fully understood from the detailed description given herein below and the accompanying drawings which are given by way of illustration only, and thus are not limitative of the present invention.

FIG. 1 is a diagram illustrating an example medical alert and monitoring system of the prior art;

FIG. 2 is a diagram illustrating a first preferred embodiment of the medical monitoring system of the present invention;

FIG. 3 is a diagram illustrating a simplified block diagram of the major component blocks of a wireless transceiver unit in a preferable embodiment of the medical monitoring system of the present invention;

FIG. 4 is an elevation view of a wearable monitoring device according to a preferred embodiment of the medical monitoring system of the present invention as applied to a wrist of an individual to be monitored;

FIG. 5 is a top plan view of the wearable monitoring device according to a preferred embodiment of the medical monitoring system of the present invention;

FIG. 6 is a side elevation view of the wearable monitoring device according to a preferred embodiment of the medical monitoring system of the present invention;

FIG. 7 is a bottom plan view of the wearable monitoring device according to a preferred embodiment of the medical monitoring system of the present invention;

FIG. 8 is an exploded view of the housing of the wearable monitoring device according to a preferred embodiment of the medical monitoring system of the present invention;

FIG. 9 is a diagram illustrating one preferred embodiment of the medical monitoring system of the present invention;

FIG. 10 is a diagram illustrating a dermal patch housing a micro-chip that forms a wearable monitoring device in accordance with another preferred embodiment of the present invention;

FIG. 11 is a diagram illustrating yet another preferable embodiment of the medical monitoring system of the present invention; and

FIG. 12 is a diagram illustrating a communication protocol utilized in an exemplary embodiment of the medical monitoring and emergency alerting system of the present invention for communication between at least one wearable monitoring device and a wireless transceiver unit.

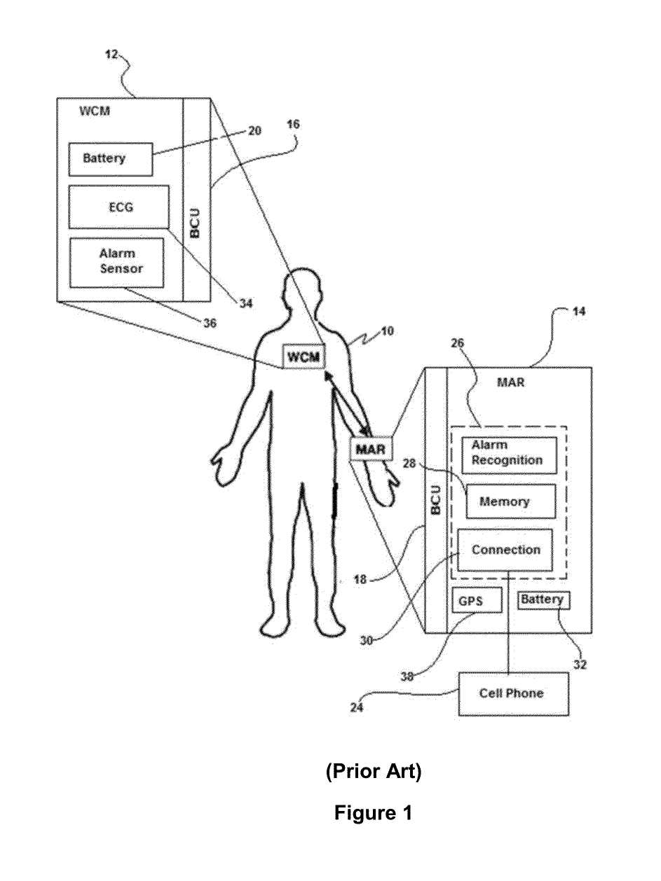

### DETAILED DESCRIPTION OF THE INVENTION

The detailed description set forth below in connection with the appended drawings is intended as a description of exemplary embodiments and is not intended to represent the only form in which the embodiments may be constructed and/or utilized. The description sets forth the functions and the sequence for constructing the exemplary embodiments. However, it is to be understood that the same or equivalent functions and sequences may be accomplished by different embodiments that are also intended to be encompassed within the scope of this disclosure.

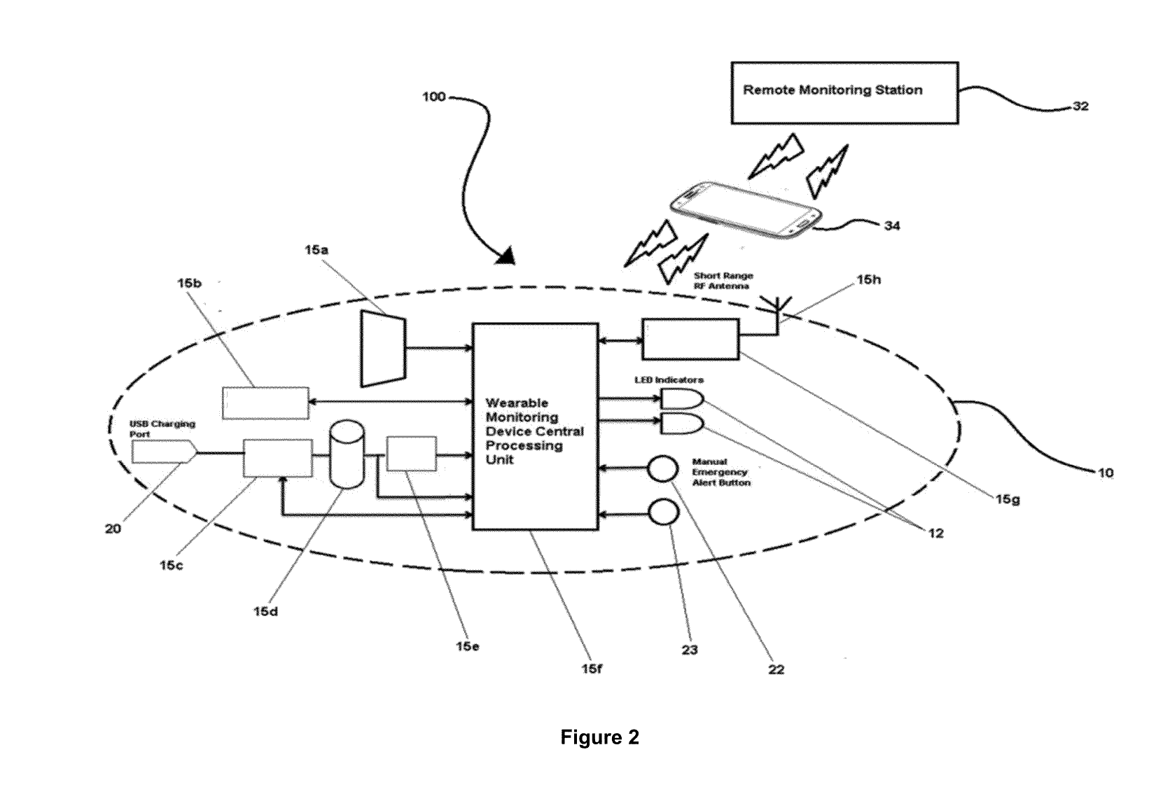

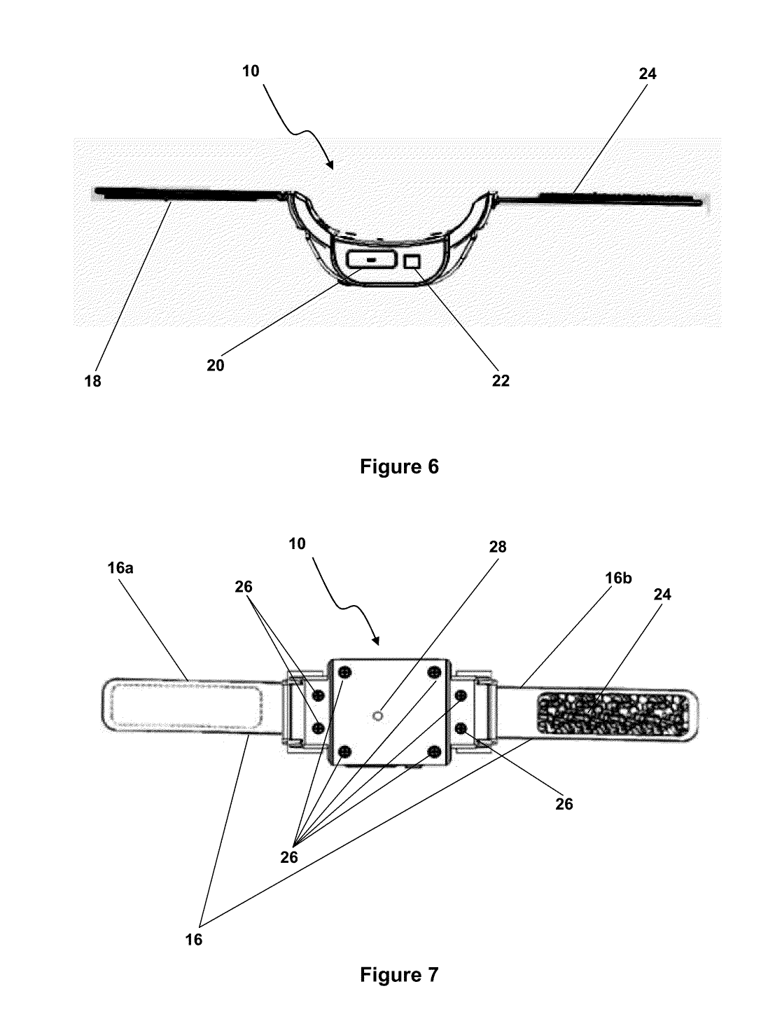

The medical monitoring system (100) of the present invention will now be described with reference to FIGS. 2 to 10.

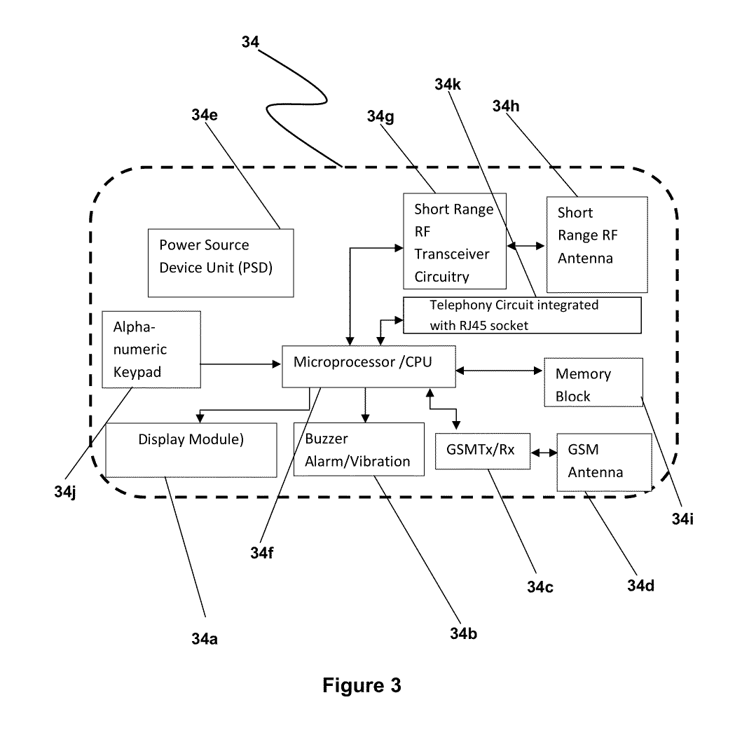

With reference to FIGS. 2 and 3, in a preferable embodiment, the present invention provides a medical monitoring system (100) for remote monitoring of an individual's physiological parameters that comprises of a wearable monitoring device (10) and a wireless transceiver unit (34).

In another preferable embodiment, the present invention provides a medical monitoring system (100) for medical monitoring and emergency alerting in relation to at least one subject, comprising of at least one wearable monitoring device (10) and a wireless transceiver unit (34).

The wearable monitoring device (10) serves to monitor a mobile subject's physiological parameters that include body temperature, perspiration and/or blood pressure analyze the physiological parameters by embodying physiological parameter measurements in the form of electrical signals and comparing the physiological parameter measurements with threshold values of physiological parameter measurements stored in a local memory (15b) of the at least one wearable monitoring device (10); produce a medical alert signal in the event the physiological parameter measurements fall beyond predetermined threshold values and further serve to wirelessly transmit the medical alert and the electrically embodied physiological parameter measurements by embodying the medical alert and the electrically embodied physiological parameter measurements in the form of RF signals to the wireless transceiver unit (34).

The wireless transceiver unit (34) is associated with the at least one subject whose physiological parameters, are being monitored and is configured to communicate with the at least one wearable monitoring device (10) via RF wireless communication, consequently enabling receipt of RF signals embodying the medical alert signal and/or RF signals embodying electrically embodied physiological parameter measurements originating from at least one wearable monitoring device (10). The wireless transceiver unit (34) and the wearable monitoring devices (10) are mutually configured to communicate with one another utilizing a bi-directional RF communication channel. More particularly, the wireless transceiver unit (34) and the wearable monitoring devices (10) are mutually, in a preferable embodiment of the medical monitoring system (100) of the present invention, configured as half-duplex RF communication devices.

The wireless transceiver unit (34), subsequently, upon receipt of a medical alert signal embodied as a RF signal, processes the received medical alert RF signal to provide a local alarm and/or provide a medical alert to a remote monitoring station (32) by way of transmitting the medical alert over a cellular network and/or a cellular network aggregated to the Internet or alternatively by way of transmitting the medical alert via a wire-line communication channel or fiber optic communication channel to the remote monitoring station (32). Similarly, the wireless transceiver unit (34) upon receipt of RF signals embodying electrically embodied physiological parameter measurements, relays the received electrically embodied physiological parameter measurements to a remote monitoring station (32) for data storage and historical trending purposes irrespective of whether a medical alert signal is received or not from the RF transmission originating from the at least one wearable monitoring device (10).

In a preferable embodiment of the medical monitoring system (100) of the present invention, a medical alert signal is produced by the wearable monitoring device (10). Alternatively in another preferable embodiment of the medical monitoring system (100) of the present invention, a medical alert signal is produced by the wireless transceiver unit (34).

In an embodiment of the medical monitoring system (100) of the present invention, RF wireless communication between the wearable monitoring device (10) and the wireless transceiver unit (34) is in accordance with the IEEE's Bluetooth Protocol.

In another embodiment of the medical monitoring system (100) of the present invention, RF wireless communication between the at least one wearable monitoring device (10) and the wireless transceiver unit (34) is in accordance with the IEEE's Zig-Bee Protocol.

In yet another embodiment of the medical monitoring system (100) of the present invention, RF wireless communication between the wearable monitoring device (10) and the wireless transceiver unit (34) is in accordance with the IEEE's 802.11 WLAN Protocol.

In yet another embodiment of the medical monitoring system (100) of the present invention, RF wireless communication between the wearable monitoring device (10) and the wireless transceiver unit (34) is achieved by transmitting and receiving wireless RF signals within frequency bands commonly used when RFID technology is deployed.

More particularly in a preferable embodiment of the medical monitoring system (100), RF wireless communication between the wearable monitoring device (10) and the wireless transceiver unit (34) is achieved by transmitting and receiving wireless RF signals within the following frequency bands:
i. 433 MHz (Ultra High Frequency Band)
ii. 868-870 MHz
iii. 902-928 MHz
iv. 2450-5480 MHz
v. 3.1-10 GHz

With reference to FIG. 2, in a preferable embodiment of the medical monitoring system (100) of the present invention, the wearable monitoring device (10) includes electronics which includes at least one physiological sensor (15a), an electrical power source (15d), an electrical power source charge management circuit (15c), a power management circuit (15e), a wearable monitoring device central processing module (15f), a wearable monitoring device memory module (15b), a wearable monitoring device RF transceiver circuit (15g) and a wearable monitoring device RF antenna (15h). In another preferable embodiment of the medical monitoring system (100) of the present invention, the wearable monitoring device (10) electronics further includes at least one display unit (12), an electrical power source charging port (20) for electrical power source charging, a power button (23) and a manual emergency alert activation button (22). In one preferable embodiment of the medical monitoring system (100) of the present invention, the electronics of the wearable monitoring device (10) are mounted on a multilayered Printed Circuit Board (15). The wearable monitoring device central processing module (15f) in addition to serving to analyze, electrically embodied physiological parameter measurements, among one of its many functions, also serves to manage charging of the electrical power source (15d) in cooperation with the electrical power source charge management circuit (15c) as well as manage the power consumed by the various electronic modules of the wearable monitoring device (10) in cooperation with the power management circuit (15e).

The wearable monitoring device memory module (15b) houses a software application executable by the wearable monitoring device central processing module (15f) to consequently enable the wearable monitoring device (10) to execute a plurality of actions that include one or more of the following actions:
i. pairing (i.e. performs a handshaking procedure to establish a bi-directional communication channel between the at least one wearable monitoring device (10) and a wireless transceiver unit (34)) the wireless transceiver unit (34) in cooperation with a software application housed within the wireless transceiver unit memory module (34i) and executed by the wireless transceiver unit central processing module (34f);
ii. acquiring at least one electrically embodied physiological parameter measurement from the at least one physiological sensor (15a);
iii. setting of customizable threshold settings of at least one physiological parameter measurement value stored within the wearable monitoring device memory module (15b);
iv. comparing of at least one electrically embodied physiological parameter measurement obtained via the physiological sensor (15a) with a threshold physiological parameter measurement value stored within the wearable monitoring device memory module (15b);
v. actuating a local alert, in the event the electrically embodied physiological parameter measurement falls beyond the threshold physiological parameter measurement value, and/or actuating a transmission of a medical alert signal to the wireless transceiver unit (34) via the wearable monitoring device RF transceiver circuit (15g) and the wearable monitoring device RF antenna (15h); and
vi. actuating the transmission of at least one electrically embodied physiological parameter measurement to the wireless transceiver unit via the wearable monitoring device RF transceiver circuit (15g) and the wearable monitoring device RF antenna (15h).

In a preferable embodiment of the medical monitoring system (100) of the present invention, the wearable monitoring device (10) includes an electrical power source (15d) which is a Li-Ion rechargeable battery or a Li-Polymer rechargeable battery.

In a preferable embodiment of the medical monitoring system (100) of the present invention, the wearable monitoring device (10) includes a removable and replaceable electrical power source (15d) which is a removable and replaceable Li-Ion rechargeable battery or a Li-Polymer battery.

In a preferable embodiment of the medical monitoring system (100) of the present invention, the electrical power source charging port (20) is a USB (Universal Serial Bus) charging port (20). In yet another preferable embodiment of the present invention, the electrical power source charging port (20) is a micro-USB (micro-Universal Serial Bus) electrical power source charging port (20).

In a preferable embodiment of the medical monitoring system (100) of the present invention, the display unit (12) comprises of a LCD display module or at least one LED (Light Emitting Diode) indicator.

In an embodiment of the medical monitoring system (100) of the present invention, the at least one wearable monitoring device (10) further includes an audible alert means which may comprise of a speaker or an electronic buzzer.

In a preferable embodiment of the medical monitoring system (100) of the present invention, the wearable monitoring device memory module (15b) of the wearable monitoring device (10) comprises of any one of or a combination of any one of a ROM (Read Only Memory) block, a RAM (Random Access Memory) block and a EEPROM (Electrically Erasable Programmable Read Only Memory) block.

In a preferable embodiment of the medical monitoring system (100) of the present invention, the wearable monitoring device (10) further includes a gyroscopic sensor and/or an accelerometer.

The physiological sensor (15a), in a preferable embodiment of the present invention includes at least one body temperature sensor that serves to enable the embodying of at least one physiological parameter measurement such as body temperature measurement, perspiration measurement (measured in terms of relative humidity) or blood pressure measurement into an electrical signal. The wearable monitoring device central processing module (15f) serves to receive/acquire the electrically embodied physiological parameter measurement from the physiological sensor(s) (15f) and determine if the electrically embodied physiological parameter measurement falls beyond predetermined thresholds. The wearable monitoring device memory module (15b) serves to store predetermined pre-set threshold values of physiological parameter measurement. The wearable monitoring device central processing module (15f), analyzes the received at least one electrically embodied physiological parameter measurement by comparing the received at least one electrically embodied physiological parameter measurement with the threshold values of the physiological parameter measurement pre-stored in the wearable monitoring device memory module (15b). If the received electrically embodied physiological parameter measurement falls beyond predetermined threshold levels, the wearable monitoring device central processing module (15f) will actuate a local alert in the event a medical alert condition is determined and transmit a medical alert signal as well as the at least one electrically embodied physiological parameter measurement to the wearable monitoring device RF transceiver circuit (15g). The wearable monitoring device RF transceiver circuit (15g) serves to receive the medical alert signal as well as the electrically embodied physiological parameter measurement transmitted by the wearable monitoring device central processing module (15f) and subsequently wirelessly transmits RF signals embodying the medical alert signal and the at least one electrically embodied physiological parameter measurement received from the wearable monitoring device central processing module (15f) to the wireless transceiver unit (34) via the wearable monitoring device RF antenna (15h).

With reference to the preceding paragraph, the wearable monitoring device central processing module (15f) will actuate a local alert in the form of a visual alert via the display unit (12) which could take the form of a message displayed on a LCD module or alternatively take the form of an LED flashing sequence of at least one LED indicator.

In one embodiment of the medical monitoring system (100) of the present invention, the at least one electrically embodied physiological parameter measurement obtained with the aid of the at least one physiological sensor (15a), is received by the wearable monitoring device central processing module (15f) which subsequently transmits the electrically embodied physiological parameter measurement to a wearable monitoring device RF transceiver circuit (15g) that serves to transmit an RF signal embodying the at least one electrically embodied physiological parameter measurement via the wearable monitoring device RF antenna (15h). The wireless transceiver unit (34) subsequently upon receipt of the RF signal embodying the electrically embodied physiological parameter measurement, processes the received RF signal embodying the electrically embodied physiological parameter measurement, to determine as to whether the electrically embodied physiological parameter measurement falls beyond predefined threshold levels or otherwise.

In a preferable embodiment of the medical monitoring system (100) of the present invention, the at least one physiological sensor (15a) of the at least one wearable monitoring device (10) comprises of a body temperature sensing element and a relative humidity sensing element.

In a preferable embodiment of the medical monitoring system (100) of the present invention, the at least one physiological sensor (15a) of the at least one wearable monitoring device (10) comprises of at least one physiological parameter sensing element.

In a preferable embodiment of the medical monitoring system (100) of the present invention, the at least one physiological sensor (15a) of the at least one wearable monitoring device (10) comprises of a plurality of sensing elements.

In an embodiment of the medical monitoring system (100) of the present invention, the at least one physiological sensor (15a) comprises of at least one sensing element which includes a body temperature sensing element.

In another preferred embodiment of the apparatus of the medical monitoring system (100) of the present invention, the at least one wearable monitoring device (10) includes a physiological sensor (15a) that comprises of a relative humidity sensing element that does not need to be in contact with the surface of the skin of a subject being monitored.

The at least one physiological sensor (15a) in this preferable embodiment of the medical monitoring system (100) of the present invention including a relative humidity sensing element that is a capacitive sensing element that enables contactless measurement of relative humidity (% RH) to provide a measure of the physiological parameter of perspiration. The advantages of utilizing a non-contact capacitive sensing element incorporated into the at least one physiological sensor (15a) of the wearable monitoring device (10) is that the non-contact sensing element inherently prevents the following:
i.) the incidence of skin irritation prevalent when contact sensors/sensing elements that have to be in direct physical contact with a subject's skin are utilized due to the fact that these sensors often include plastic and metal electrodes that may cause the skin of the subject being monitored to be irritable;
ii.) the incidence of skin irritation prevalent when contact sensors/sensing elements that have to be in direct physical contact with a subject's skin are utilized due to the fact that these sensors often involve the flow of current or the imposition of a voltage on the surface of the skin of a subject being monitored; and
iii.) the incidence of measurement errors due to corrosion of electrodes in the case of contact based physiological sensors/sensing elements, caused by the subject's perspiration.

The reader should be informed that the above is just a non-exhaustive list of advantages derived from the use of a non-contact capacitive sensing element incorporated into the at least one physiological sensor (15a) of the wearable monitoring device (10) in a preferable embodiment of the medical monitoring system (100) of the present invention.

The reader should take note that the at least one physiological sensor (15a) that incorporates the aforementioned non-contact capacitive sensing element in a preferable embodiment of the medical monitoring system (100) of the present invention, is a sensor obtained from any one of the range of Honeywell Humidlcon™ Digital Humidity/Temperature Sensors:HIH6130/6131 Series.

The provision of a manual emergency alert activation button (22) in the at least one wearable monitoring device (10) in a preferable embodiment of the medical monitoring system (100) of the present invention, enables a subject wearing the wearable monitoring device (10) to actuate the transmission of a medical alert to a remote monitoring station (32) via the wireless transceiver unit (34). More particularly, upon a subject pressing the manual emergency alert activation button (22), the wearable monitoring device central processing module (15f) will detect the depression of the manual emergency activation button (22) and subsequently generate a medical alert signal and further initiate transmission of the medical emergency alert signal to the wireless transceiver unit (34) via the wearable monitoring device RF transceiver circuit (15g) and the wearable monitoring device RF antenna (15h). The wireless transceiver unit (34) subsequently produces a local alert, and relays the medical emergency alert signal to a remote monitoring station (32).

The provision of a manual emergency alert activation button (22) in the wearable monitoring device (10) in the preferred embodiment of the medical monitoring system (100) as described in the preceding paragraph, further provisions for a local alert at the wearable monitoring device (10) in the form of a visual alert via the at least one display unit (12) which takes the form of a message displayed on a LCD module or alternatively takes the form of an LED flashing sequence of at least one LED indicator.

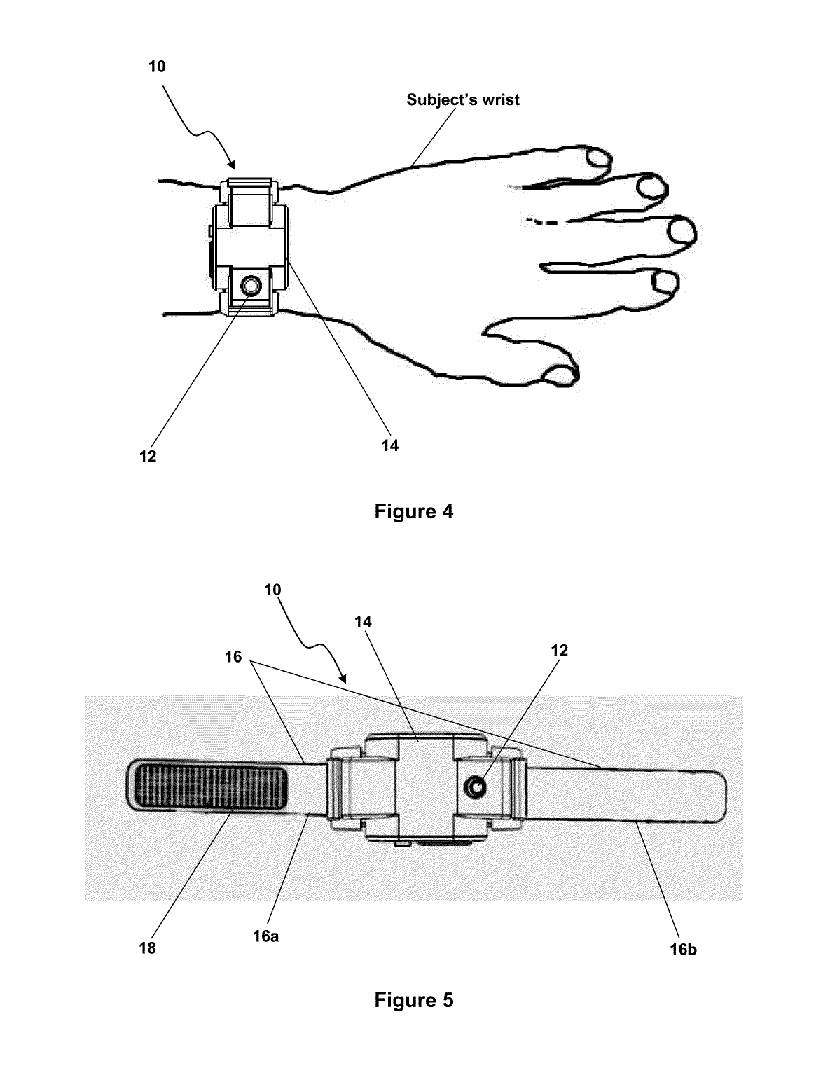

With reference to FIGS. 4 to 8, in a preferable embodiment of the medical monitoring system (100) of the present invention, the wearable monitoring device (10) takes the form of a housing (14) securable to a pair of straps (16) with electronics mounted on a multi-layered PCB (Printed Circuit Board) (15) housed in the housing (14). More particularly, in this preferable embodiment of the present invention, the at least one wearable monitoring device (10) comprises of a housing (14) comprising of a top casing (14a) and a bottom casing (14b) that are respectively substantially curved at longitudinally oriented symmetrically opposing ends (14c, 14g), and a pair elastomeric wrist straps (16), a first elastomeric wrist strap (16a) and a second elastomeric wrist strap (16b). Each elastomeric wrist strap (16a, 16b) of the pair elastomeric wrist straps (16) are rotatably attached to each longitudinally oriented substantially curved symmetrically opposing end (14c) of the top casing (14a) of the housing (14) of the at least one wearable monitoring device (10).

In a preferred embodiment of the medical monitoring system (100) of the present invention, the pair of elastomeric wrist straps (16a, 16b), are fabricated from a hypoallergenic elastomer.

In another preferred embodiment of the medical monitoring system (100) of the present invention, the at least one wearable monitoring device (10) takes the form of a housing (14) securable to a strap fabricated from a hypoallergenic elastomer.

The electronics mounted on the multi-layered PCB (Printed Circuit Board) (15) including at least one physiological sensor (15a), a wearable monitoring device central processing module (15f), a wearable monitoring device memory module (15b), a wearable monitoring device RF transceiver circuit (15g), a wearable monitoring device RF antenna (15h), a display unit (12), an electrical power source charging port (20) and a manual emergency activation button (22).

The multilayered PCB (Printed Circuit Board) (15) including a plurality of perforations to facilitate securing of the multilayered PCB (15) onto the bottom casing (14b) of the housing (14) of the wearable monitoring device (10) in a preferable embodiment.

With reference to FIGS. 5 to 7, the first elastomeric wrist strap (16a) of the pair of elastomeric straps (16) includes a hook fabric strip (18) and the second elastomeric wrist strap (16b) of the pair of elastomeric wrist straps (16) includes a loop fabric strip (24).

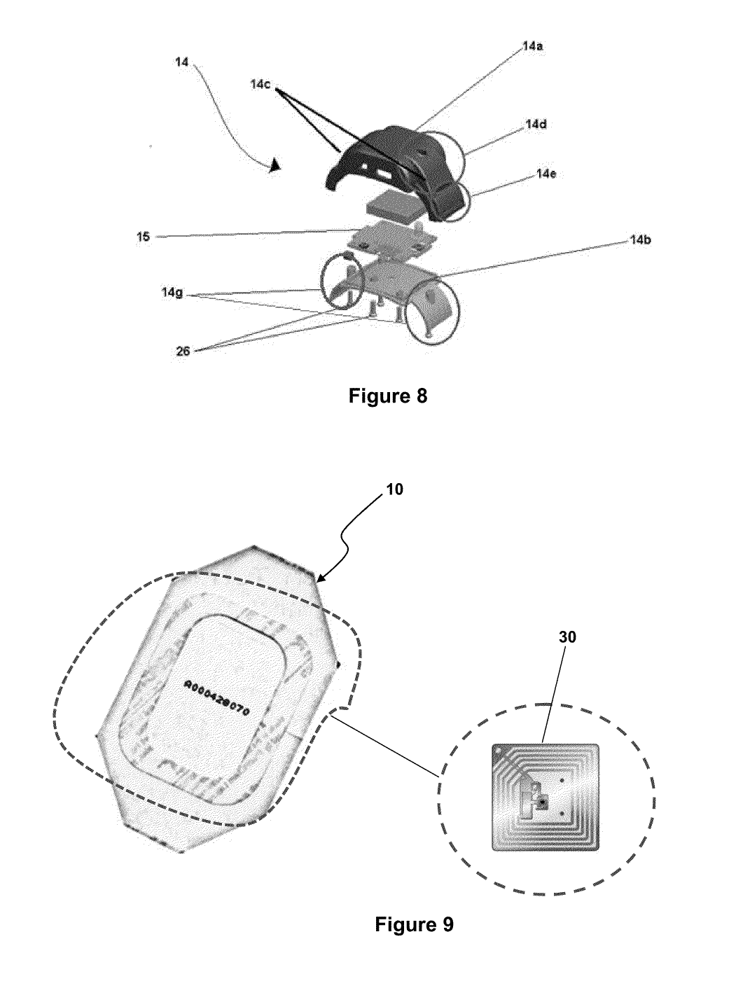

With reference to FIGS. 7 and 8, the bottom casing (14b) comprises a plurality of threaded bore-holes that serve to enable the threading of a plurality of screws (26) respectively. The screws (26) serve to secure the printed circuit board (PCB) (15) to the bottom casing (14b) of the housing (14). The multilayered printed circuit board (PCB) (15) is secured to the bottom casing (14b) of the housing (14) by aligning the plurality of threaded bore-holes of the bottom casing (14b) to the plurality of perforations of the multilayered PCB (15) and threading a plurality of screws (26) of appropriate size and with appropriate threads through the combination of the plurality of bore holes of the bottom casing (14b) and the corresponding plurality of perforations of the multilayered PCB (15).

In addition to the plurality of bore holes, the bottom casing (14b) of the housing (14) further includes at least one perforation (28) that serves to provide an opening through which the physiological sensor (15a) which, in a preferable embodiment of the present invention, incorporates a capacitive sensing element that enables contactless measurement of relative humidity (% RH), to sample and measure relative humidity from an area in the immediate vicinity on the surface of the skin of the subject's wrist.

With reference to FIG. 8, the top casing (14a) includes substantially curved symmetrically opposing ends (14c) that project away from the housing (14). Each longitudinally oriented substantially curved symmetrically opposing end (14c) of the top casing (14a) includes a pair of substantially curved projections (14d, 14e), a first upper substantially curved projection (14d) and a second lower substantially curved projection (14e). The pair of substantially curved projections (14d, 14e) serving to deflect air drafts from the vertical/horizontal and diagonal directions, to prevent transient air movement or air drafts from affecting the reading of the at least one physiological sensor (15a) of the wearable monitoring device (10) which in this preferred embodiment of the medical monitoring system (100) is a capacitive sensor for the measurement of relative humidity (% RH).

With reference to FIG. 4, the wearable monitoring device (10) that takes the form of a wrist watch like device in a preferable embodiment of the medical monitoring system (100) of the present invention as described in the immediately preceding paragraphs above, is worn by first placing the housing (14) which houses the printed circuit board (PCB) (15), such that the top face of the housing (14) appears to rests on the subject's wrist (when viewed from the top). Subsequently the first and second elastomeric wrist straps (16a, 16b) are wound around the wrist of the subject, such that the hook fabric strip (18) of the first elastomeric wrist strap (16a) is positioned facing and is consequently coupled to the loop fabric strip (24) of the second elastomeric wrist strap (16b), to thus consequently firmly attach the wearable monitoring device (10) onto a subject's wrist.

With reference to FIG. 3, in a preferable embodiment of the medical monitoring system (100) of the present invention, the wireless transceiver unit (34) includes a first RF antenna (34h), a first RF transceiver circuit (34g), a wireless transceiver unit central processing module (34f), a wireless transceiver unit memory module (34i), a second RF transceiver circuit (34c), a second RF antenna (34d), a display module (34a), an electronic buzzer (34b), an alphanumeric keypad module (34j) and a wire-line communication module (integrated with a wire-line communication socket, i.e. which may in a preferable embodiment include a RJ45 socket) (34k).

The wireless transceiver unit memory module (34i) houses a software application executable by the wireless transceiver unit central processing module (34f) to consequently enable the wireless transceiver unit to execute a plurality of actions that include one or more of the following actions:
i.) pairing (i.e. performs a handshaking procedure to establish a bi-directional communication channel between at least one wearable monitoring device (10) and a wireless transceiver unit (34)) of the least one wearable monitoring device to the wireless transceiver unit;
ii.) setting of customizable threshold settings of at least one physiological parameter measurement value stored within the wireless transceiver unit memory module (34i);
iii.) acquiring a wirelessly transmitted RF signal embodying a medical alert signal and/or a RF signal embodying at least one electrically embodied physiological parameter measurement via the first RF antenna (34h) and the first RF transceiver circuitry (34g)
iv.) comparing of a received at least one electrically embodied physiological parameter measurement with a threshold physiological parameter measurement value;
v.) actuation of a local alert at the wearable monitoring device and/or at the wireless transceiver unit and/or actuating a transmission of a medical alert signal to a remote monitoring station via any one of or a combination of any one of a wireless communication channel, a fiber-optic communication channel or a wire-line communication channel; and
vi.) actuating a transmission of a received at least one electrically embodied physiological parameter measurement to a remote monitoring station via any one of or a combination of any one of a wireless communication channel, a fiber optic communication channel or a wire-line communication channel.

In a preferred embodiment of the medical monitoring system (100) of the present invention, the wireless transceiver unit's first RF antenna (34h) is an omni-directional antenna.

In a preferred embodiment of the medical monitoring system (100) of the present invention, the wireless transceiver unit memory module (34i) of the wireless transceiver unit (34) comprises of any one of or a combination of any one of a ROM (Read Only Memory) block, a RAM (Random Access Memory) block and a EEPROM (Electrically Erasable Programmable Read Only Memory block.

With reference FIGS. 2 and 3, in a preferable embodiment of the medical monitoring system (100) of the present invention, the wireless transceiver unit (34) serves to wirelessly receive via the first RF antenna (34h), an RF signal embodying at least one electrically embodied physiological parameter measurement transmitted from the wearable monitoring device (10) via the wearable monitoring device first RF antenna (15h). The received RF signal that embodies at least one electrically embodied physiological parameter measurement, is received via the wireless transceiver unit's first RF antenna (34h) and is subsequently down-converted and demodulated by the wireless transceiver unit's first RF transceiver circuit (34g) to provide the at least one electrically embodied physiological parameter measurement signal (as originally obtained by the at least one physiological sensor (15a) of the at least one wearable monitoring device (10) that is subsequently transmitted to the wireless transceiver unit central processing module (34f). The wireless transceiver unit central processing module (34f), serving to analyze the received electrically embodied physiological parameter measurement signal by comparing with threshold values of physiological parameter measurement pre-stored in the wireless transceiver unit memory module (34i). In the event, that the received electrically embodied physiological parameter measurement falls beyond the predetermined physiological parameter measurement threshold values, the wireless transceiver unit central processing module (34f), determines a medical alert condition and will proceed to transmit a medical alert signal as well as receive at least one electrically embodied physiological parameter measurement to the wireless transceiver unit second RF transceiver circuit (34c). The wireless transceiver unit second RF transceiver circuit (34c), upon receipt of the medical alert signal and the at least one electrically embodied physiological parameter measurement, will subsequently embody the medical alert signal and the at least one electrically embodied physiological parameter measurement as RF signals and transmit the RF signals embodying the medical alert signal and the at least one electrically embodied physiological parameter measurement to a remote monitoring station (32) via the second RF antenna. The medical alert signal is transmitted to the remote monitoring station (32) for medical emergency alerting to alert a remote care-giver or alert a medical emergency response team. The at least one electrically embodied physiological parameter measurement is transmitted to the remote monitoring station (32) for data storage and historical trending purposes.

In addition, with reference to the immediately preceding paragraph, in this preferable embodiment of the medical monitoring system (100) of the present invention, upon determination of a medical alert condition, the wireless transceiver unit central processing module (34f) will initiate a further transmission of a medical alert signal to the at least one wearable monitoring device (10) via the wireless transceiver unit first RF transceiver circuit (34g) and the wireless transceiver unit first RF antenna (34h). The medical alert signal will be embodied as an RF signal by the wireless transceiver unit first RF transceiver (34g) and is transmitted via the wireless transceiver unit first RF antenna (34h) to the at least one wearable monitoring device (10) which will receive the RF signal embodying the medical alert signal via the wearable monitoring device RF antenna (15h) and the wearable monitoring device RF transceiver circuit (15g). The wearable monitoring device RF transceiver circuit (15g) will subsequently recover the originally transmitted medical alert signal as originally transmitted by the wireless transceiver unit (34) and transmit the recovered medical alert signal to the wearable monitoring device central processing module (15f). The wearable monitoring device central processing module (15f) will subsequently, upon receipt of the medical alert signal, actuate a local alert in the form of a visual alert via the at least one display unit (12) which could take the form of a message displayed on a LCD module or alternatively take the form of an LED flashing sequence of at least one LED indicator.

Again with reference to FIGS. 2 and 3, in another preferable embodiment of the medical monitoring system (100) of the present invention, the wireless transceiver unit (34) serves to wirelessly receive via the first RF antenna (34h), RF signals embodying a medical alert signal and at least one electrically embodied physiological parameter measurement transmitted from the wearable monitoring device (10) via the wearable monitoring device RF antenna (15h). Upon receipt of the RF signals embodying the medical alert signal and the at least one electrically embodied physiological parameter measurement by the wireless transceiver unit first RF antenna (34h), the RF signals embodying the medical alert signal and the at least one physiological parameter measurement, propagate to the wireless transceiver unit first RF transceiver circuit (34g). The wireless transceiver unit first RF transceiver circuit (34g), subsequently down-converts and demodulates the received RF signals embodying the medical alert signal and the at least one electrically embodied physiological parameter measurement and subsequently recovers the medical alert signal as well as the at least one electrically embodied physiological parameter measurement that were originally generated at the at least one wearable monitoring device (10). The recovered medical alert signal and at least one electrically embodied physiological parameter measurement is subsequently transmitted to the wireless transceiver unit central processing module (34f), which subsequently actuates the transmission of the medical alert signal and the at least one electrically embodied physiological parameter measurement to the wireless transceiver unit second RF transceiver circuit (34c). The wireless transceiver unit second RF transceiver circuit (34c), upon receipt of the medical alert signal and the at least one electrically embodied physiological parameter measurement, will subsequently embody the medical alert signal and the at least one electrically embodied physiological parameter measurement in the form of RF signals and transmit the RF signals embodying the medical alert signal and the at least one electrically embodied physiological parameter measurement to a remote monitoring station (32). The medical alert signal is transmitted to the remote monitoring station (32) for medical emergency alerting to alert a remote care-giver or alert a medical emergency response team. The at least one electrically embodied physiological parameter measurement is transmitted to the remote monitoring station (32) for data storage and historical trending purposes.

The remote monitoring station (32) in a preferable embodiment of the medical monitoring system of the present invention is a cellular phone of a remote care-giver. The remote monitoring station (32) in another preferable embodiment of the medical monitoring system of the present invention is a remote call center of a medical emergency response team.

With reference to the preceding four paragraphs above, in accordance with a preferable embodiment of the medical monitoring system (100) of the present invention, in the event the remote monitoring station (32) is cellular phone of a remote care-giver, the data storage of the at least one electrically embodied physiological parameter measurement is storage of the at least one electrically embodied physiological parameter measurement in memory of the remote care-giver's cellular phone. In event the remote monitoring station (32) is a call-center of a medical emergency response team, the data storage of the at least one electrically embodied physiological parameter measurement is storage of the at least one electrically embodied physiological parameter measurement in a database of the call center.

In a preferable embodiment of the medical monitoring system (100) of the present invention, the medical alert signal transmitted to the remote monitoring station (32) is an SMS alert message embodied as an RF signal.

In another preferable embodiment of the medical monitoring system (100) of the present invention, the medical alert signal is a call signal to a predetermined number of a remote monitoring station (32). In other words, the wireless transceiver unit central processing module (34f) will initiate a telephone call to a predetermined number of a remote monitoring station (32).

With reference to FIGS. 2 and 3, in a preferable embodiment of the medical monitoring system (100) of the present invention, the wireless transceiver unit (34) is a cellular phone (34A) associated with the subject whose physiological parameters are to be monitored that includes a first RF antenna (34h), a first RF transceiver circuit (34g), a wireless transceiver unit central processing module (34f), a memory module (34i), a second RF transceiver circuit (34c), a second RF antenna (34d), a display module (34a), an electronic buzzer (34b), an alphanumeric keypad module (34j).

Again with reference to FIG. 3, in a preferable embodiment of the medical monitoring system (100) of the present invention, the wireless transceiver unit (34) includes a wire-line communication module integrated with a wire-line communication socket (34k) (that may in a preferable embodiment include a RJ45 socket), thus enabling the wireless transceiver unit (34) to transmit a medical alert signal to a remote monitoring station (32) by way of a wire-line communication channel. More particularly the provision of a wire-line communication module integrated with a wire-line communication socket (that may in a preferable embodiment include a RJ45 socket) (34k) within the wireless transceiver unit (34) in a preferable embodiment of the medical monitoring system (100) of the present invention, enables the transmission of a medical alert signal by the wireless transceiver unit (34) to a remote monitoring station (32) via a PSTN (Public Switched Telephone Network) through the POTs (Plain Old Telephone Service).

In yet another preferable embodiment of the medical monitoring system (100) of the present invention, the wireless transceiver unit (34) includes a fiber-optic communications module (not shown) that enables the conversion of electrical signals to optical signals and vice-versa to thus enable transmission and reception of information embodied as electrical signals as optical signals to or from a remote monitoring station (32) via a fiber optic communication network, a fiber optic communication network aggregated to the internet and/or to a PSTN (Public Switched Telephone Network). More specifically the wireless transceiver unit (34) includes a fiber-optic communications module (not shown) that enables conversion of electrical signals that include a medical alert signal and at least one electrically embodied physiological parameter measurement into optical signals for transmission to a remote monitoring station (32) via a fiber optic communication network, a fiber optic network aggregated to the internet and/or to a PSTN.

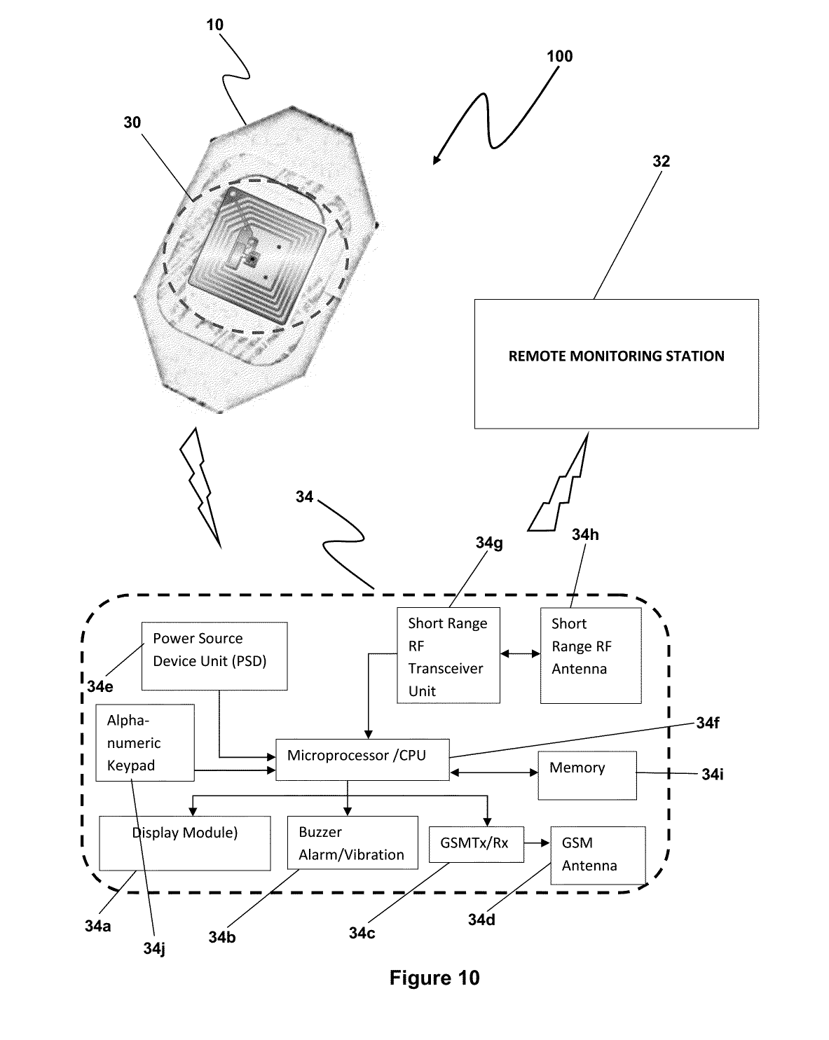

With reference to FIGS. 9 and 10, in a preferable embodiment of the medical monitoring system (100) of the present invention, the wearable monitoring device (10) takes the form of a dermal patch (10) which comprises of electronics formed on a single micro-chip (30) which include RFID circuitry and at least one sensing element. The at least one sensing element enabling the embodying of at least one physiological parameter measurement such as body temperature measurement, measurement of perspiration (i.e. in terms of relative humidity measurement) or blood pressure into at least one electrically embodied physiological parameter measurement. The wearable monitoring device (10) in this preferable embodiment including electronic circuitry that enables transmission of the at least one electrically embodied physiological parameter measurement (by embodying the electrically embodied physiological parameter measurement as RF signals) to the wireless transceiver unit (34) for further processing.

In a preferable embodiment of the medical monitoring system (100) of the present invention, the threshold values of the at least one physiological parameter measurement stored in either the wearable monitoring device memory module (15b) of the wearable monitoring device (10) or the wireless transceiver unit memory module (34i) of the wireless transceiver unit (34), are customizable to accommodate the age, gender, normal medical condition of a subject being monitored and the time of the day (to accommodate the amount and type of activities that a subject may undertake during particular time periods within a day).

With reference to FIGS. 9 and 10, in a preferable embodiment of the medical monitoring system (100) of the present invention, the wearable monitoring device (10) takes the form of a dermal patch (10) that houses a single integrated circuit (30) with electronics which include at least one physiological sensor (15a), a wearable monitoring device central processing module (15f), a wearable monitoring device memory module (15b), a wearable monitoring device RF transceiver circuit (15g) and a wearable monitoring device RF antenna (15h). More particularly, the electronics which include the at least one physiological sensor (15a), the wearable monitoring device central processing module (15f), the wearable monitoring device memory module (15b), the wearable monitoring device RF transceiver circuit (15g) and the wearable monitoring device RF antenna (15h) are integrated into a single integrated circuit (30).

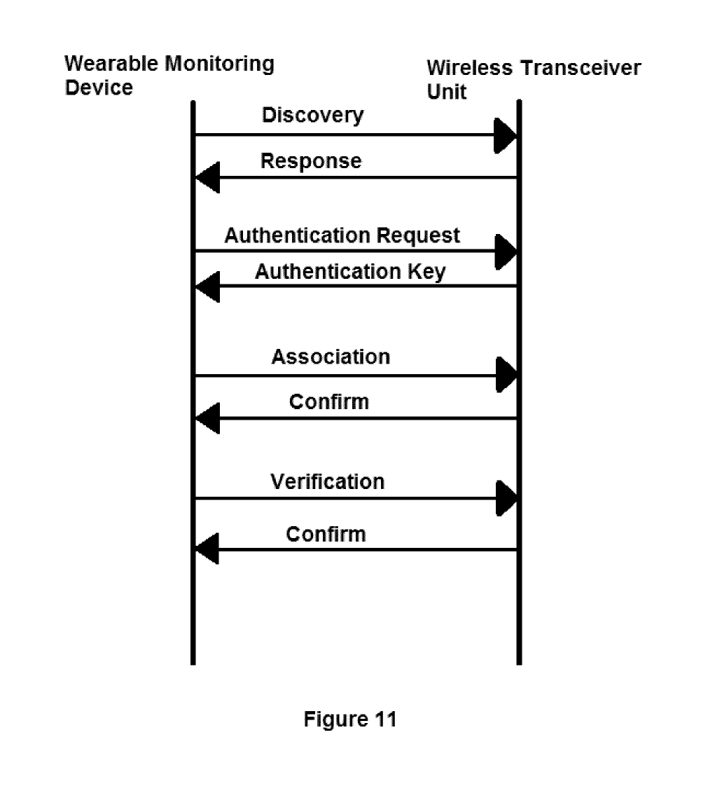

With reference to FIG. 11, in yet another preferable embodiment of the medical monitoring system (100) of the present invention, the wireless transceiver unit (34) is a wireless network access device (34B) that includes a first RF antenna (34h), a first RF transceiver circuit (34g), a central processing unit (34f), a memory module (34i), and a wire-line communication module and/or a fiber-optic communication module.

With reference to FIG. 11, in a preferable embodiment of the medical monitoring system (100) of the present invention, as described in the immediately above paragraph, the at least one wearable monitoring device (10) transmits at least one electrically embodied physiological parameter measurement and/or a medical alert signal to the wireless network access device (34B) that serves to wirelessly receive the at least one electrically embodied physiological parameter measurement and/or medical alert signal and transmit the at least one electrically embodied physiological parameter measurement and/or a medical alert signal to a remote monitoring station (32) which comprises of a central computer (32a) and a main-frame server (32b), via a wire-line communication channel or a fiber-optic communication channel.

The central computer (32a) of the remote monitoring station (32) serving as an operator console that includes hardware capable of receiving the medical alert or at least one electrically embodied physiological parameter measurement and further includes software applications that enable the analysis of the received at least one electrically embodied physiological parameter measurement and actuation of medical alert to a team of emergency response personnel including doctors and nurses upon receipt of a medical alert signal. The central computer (32a) further serving to store the received at least one electrically embodied physiological parameter measurement in a main-frame server (32b).

With reference to FIG. 11, in a preferable embodiment of the medical monitoring system (100) of the present invention, a plurality of wearable monitoring devices (10) associated with a plurality of subjects each communicate by way of bidirectional RF communication with the wireless network access device (34B). Each of the plurality of wearable monitoring devices (10) communicating with the wireless network access (34B) by way of RF transmissions that include unique authentication keys that serve to distinguish and identify between transmitting wearable monitoring devices (10).

The embodiment of the medical monitoring system (100) as illustrated in FIG. 11 finding utility in medical institutions such as medical centers or hospitals wherein medical care staff including nurses and doctors need to constantly be able to keep tabs on subjects despite not being in the immediate vicinity of the subjects.

In the description of a preferable embodiment of the medical monitoring system (100) as described in any one of the preceding paragraphs above, the at least one wearable monitoring device (10) periodically transmits an RF signal embodying the at least one electrically embodied physiological parameter measurement to the wireless transceiver unit (34). The wireless transceiver unit (34) upon periodically receiving the RF signal embodying the at least one electrically embodied physiological parameter measurement, recovers the at least one electrically embodied physiological parameter measurement as originally generated at the at least one wearable monitoring device (10) and periodically relays the at least one electrically embodied physiological parameter measurement by way of long range RF transmission to a remote monitoring station (32) which provisions for the storage of the at least one electrically embodied physiological parameter measurement for historical trending purposes.

In one preferable embodiment of the medical monitoring system (100), the at least one wearable monitoring device (10) continuously transmits an RF signal embodying the at least one electrically embodied physiological parameter measurement to the wireless transceiver unit (34). Similarly, the wireless transceiver unit (34) upon continuously receiving the RF signal embodying the at least one electrically embodied physiological parameter measurement, recovers the at least one electrically embodied physiological parameter measurement as originally generated at the at least one wearable monitoring device (10) and continuously relays the at least one electrically embodied physiological parameter measurement by way of long range RF transmission to a remote monitoring station (32) which provisions for the storage of the at least one electrically embodied physiological parameter measurement for historical trending purposes.

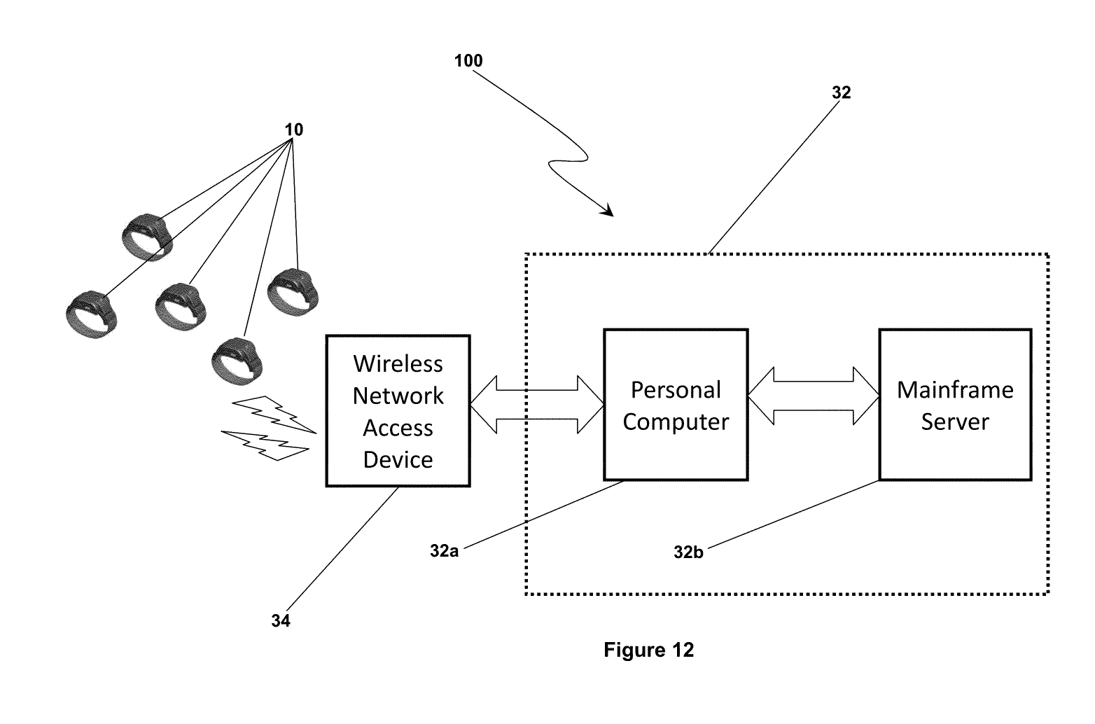

In the description of the medical monitoring system (100) as described in any one of the preceding paragraphs above, the at least one wearable monitoring device (10) performs a hand-shaking procedure to establish a bi-directional communication channel (i.e. pairing) between the at least one wearable monitoring device (10) and the wireless transceiver unit (34). With reference to FIG. 12, in an exemplary embodiment of the medical monitoring system (100) of the present invention, in order to establish a communication channel between the at least one wearable monitoring device (10) and the wireless transceiver unit (34), the at least one wearable monitoring device (10) transmits a discovery signal to the wireless transceiver unit (34). A response signal is transmitted back to the at least one wearable monitoring device (10) by the wireless transceiver unit (34). Security can be set up by exchanging a shared authentication key between the wearable monitoring device (10) and the wireless transceiver unit (34). An authentication request is communicated from the at least one wearable monitoring device (10) and to the wireless transceiver unit (34) and an authentication key is returned by the wireless transceiver unit (34) to the at least one wearable monitoring device (10). An association signal is sent from the wearable monitoring device (10) to the wireless transceiver unit (34) and the wireless transceiver unit (34) returns a confirm signal to verify establishment of a connection between the at least one wearable monitoring device (10) and the wireless transceiver unit (34).

With reference to the exemplary embodiment of the medical monitoring system (100) of the present invention as described in the preceding paragraph immediately above, once communication is established, a verification signal is sent at various times from the at least one wearable monitoring device (10) to the wireless transceiver unit (34). When a verification signal is received, the wireless transceiver unit (34) returns a confirm signal to indicate that the communication channel between the wireless transceiver unit (34) and the at least one wearable monitoring device (34) is active.

With reference to the immediately preceding two paragraphs, in relation to the at least one wearable monitoring device (10), the various stages of the handshaking from “discovery” to association is indicated by a visual indication on a display unit (12) of the at least one wearable monitoring device (10), which may take the form of a pattern of LED blinking sequence in a preferable embodiment of the medical emergency alert and monitoring system (100) of the present invention.

In one aspect of the medical monitoring system (100) of the present invention, there is provided a method to determine and alert a subject wearing a wearable monitoring device (10) that is configured to measure at least one physiological parameter, as to whether the wearable monitoring device (10) is in firm contact with the subject's skin utilizing an electrically embodied physiological parameter measurement obtained by an at least one physiological sensor (15a) of the wearable monitoring device (10); the method being embodied as a software application residing within the wireless transceiver unit memory module (34i) of the wireless transceiver unit (34) and/or within the wearable monitoring device memory module (15b) of the wearable monitoring device (10) and comprising the steps of:
i. acquiring the electrically embodied physiological parameter;
ii. determining if the electrically embodied physiological parameter measurement acquired in step (i) falls beyond a predetermined range of measurements that represent the normal range of measurement values when the wearable monitoring device (10) is tightly coupled to a subject's skin; and
iii. actuating an audible or visual alarm in the event the electrically embodied physiological parameter measurement exceeds the normal range of measurement values;

In a preferable embodiment of the medical monitoring system (100) of the present invention, the method to determine and alert a subject, as to whether the at least one wearable monitoring device (10) is in firm contact with a subject's skin utilizing the at least one electrically embodied physiological parameter measurement obtained by the at least one physiological sensor (15a) of the at least one wearable monitoring device (10) utilizes a body temperature measurement of a subject.

More particularly, in a preferable embodiment of the medical monitoring system (100) of the present invention, the method to determine and alert a subject wearing a wearable monitoring device (10) that is configured to measure at least one physiological parameter, as to whether the wearable monitoring device (10) is in firm contact with the subject's skin utilizing an electrically embodied physiological parameter measurement which includes body temperature measurement obtained by an at least one physiological sensor (15a) of the wearable monitoring device (10), comprises of the following steps:
i. acquiring the electrically embodied physiological parameter which includes body temperature measurement;
ii. determining if the electrically embodied physiological parameter measurement which includes body temperature measurement acquired in step (i) falls beyond a predetermined range of measurements that represent the normal range of measurement values when the wearable monitoring device (10) is tightly coupled to the subject's skin; and
iii. actuating an audible or visual alarm in the event the at least one electrically embodied physiological parameter measurement which includes body temperature measurement exceeds the normal range of measurement values;

With relation to the method to determine and alert a subject, as to whether the at least one wearable monitoring device (10) is in firm contact with a subject's skin as described in the preceding paragraphs above, in one embodiment of the medical monitoring system (100) of the present invention, the audible or visual alarm is generated at the at least one wearable monitoring device (10). The visual alarm at the at least one wearable monitoring device (10) may take the form of a visual alert via the at least one display unit (12) which could take the form of a message displayed on a LCD module or alternatively take the form of an LED flashing sequence of at least one LED indicator. In one embodiment of the medical monitoring system (100) of the present invention, the audible or visual alarm is generated at the wireless transceiver unit (34). The visual alarm at the wireless transceiver unit (34) may take the form of a visual alert via the display module (34a).

In another aspect of the medical monitoring system (100) of the present invention there is provided a method to alert a remote care-giver or a remote team of medical emergency response personnel via a remote monitoring station (32) through the wireless transceiver unit (34) that is in communication with the at least one wearable monitoring device (10) and the remote monitoring station (32), in the event a medical emergency condition is determined. The method being embodied by a software application residing within the wireless transceiver unit memory module (34i) of the wireless transceiver unit (34) and comprises the steps of:
i. acquiring at least one electrically embodied physiological parameter measurement from a wearable monitoring device (10) worn by a subject who is being monitored;
ii. determining if the at least one electrically embodied physiological parameter measurement acquired in step (i) falls beyond a predetermined range of measurements that represent the normal range of physiological parameter measurement values when the subject is healthy;
iii. actuating a local alarm on the wearable monitoring device (10);
iv. automatically executing a phone call to at least one predetermined primary number corresponding to at least one remote monitoring station (32) which may include a cell-phone of a remote care-giver or a remote call center of a medical emergency response team; the phone call being executed a predetermined number of times until it is acknowledged by the remote monitoring station (32);
v. automatically executing a phone call to at least one alternate predetermined secondary number corresponding to at least one remote monitoring station (32) which may include a cell phone of a remote care-giver or a remote call center of a medical emergency response team in the event, the phone call to the predetermined primary number is not acknowledged;
vi. repeating steps (iv) and (v) until either one of the phone calls to the at least one predetermined primary number or the at least one predetermined secondary number is acknowledged; and
vii. automatically executing the wireless transmission of an SMS alert to at least one predetermined primary or secondary number corresponding to at least one remote monitoring station (32) which may include a cell-phone of a remote care-giver or a remote call center of a medical emergency response team.

As mentioned in a preceding paragraph, in a preferable embodiment of the medical monitoring system (100) of the present invention, the medical alert signal transmitted to the remote monitoring station (32) is an SMS alert message embodied as an RF signal. In one embodiment of the medical monitoring system (100) of the present invention, the SMS alert message is a user defined, customizable SMS alert message.

In a preferable embodiment of the medical monitoring system (100) of the present invention, there is provided a method to alert a remote care-giver or a remote team of medical emergency response personnel via a remote monitoring station (32) through the wireless transceiver unit (34) that is in communication with the wearable monitoring device (10) and the remote monitoring station (32). The method being embodied and executed by a software application residing in the wireless transceiver unit memory module (34i) of the wireless transceiver unit (34) and comprises the steps of:
i. acquiring at least one electrically embodied physiological parameter from a wearable monitoring device (10) worn by a subject who is being monitored;
ii. determining if the at least one electrically embodied physiological parameter measurement acquired in step (i) falls beyond a predetermined range of measurements that represent the normal range of physiological parameter measurements when the subject is healthy;
iii. actuating a local alarm on the wearable monitoring device (10);
iv. automatically executing a phone call to one predetermined primary number corresponding to at least one remote monitoring station (32) which may include a cell-phone of a remote care-giver or a remote call center of a medical emergency response team; the phone call being executed a predetermined number of times until it is acknowledged by the remote monitoring station (32);
v. automatically executing a phone call to at least one other predetermined alternate number corresponding to at least one remote monitoring station (32) which may include a cell phone of a remote care-giver or a remote call center of a medical emergency response team for a predetermined number of times in the event, the phone call to the one predetermined primary number is not acknowledged after being executed for a predetermined number of times;
vi. repeating steps (iv) and (v) until either one of the phone calls to the at least one predetermined primary number or the at least one predetermined secondary number is acknowledged; and
vii. automatically executing a wireless transmission of an SMS alert to the one predetermined number or the at least one other predetermined alternate number corresponding to at least one remote monitoring station (32) which may include a cell-phone of a remote care-giver or a remote call center of a medical emergency response team.

In a preferable embodiment of the medical monitoring system (100) of the present invention, upon successful receipt and engaging of a phone call or successful receipt of an SMS alert, the remote monitoring station (32) which may include a remote care-giver's cellular phone or a remote call center of a medical emergency response team, will transmit an acknowledgement signal to the wireless transceiver unit (34) which will subsequently transmit an acknowledgement signal to the at least one wearable monitoring device (10). In a preferable embodiment of the present invention, the wireless transceiver unit (34) will provide a visual indication of the received acknowledgement signal received from the remote monitoring station (34). In another preferable embodiment of the present invention, the at least one wearable monitoring device (10) will provide a visual indication of the received acknowledgement signal received via the wireless transceiver unit (34) originating from the remote monitoring station (32), the visual indication taking the form of a message displayed on a LCD module or alternatively taking the form of a LED flashing sequence of at least one LED indicator.

In a preferable embodiment of the medical monitoring system of the present invention, the wearable monitoring device includes a software application residing within either the wearable monitoring device memory module (15b) of the wearable monitoring device (10) or the wireless transceiver unit memory module (34i) of the wireless transceiver unit (34), that in cooperation with a gyroscopic sensor and/or an accelerometer enables the detection of an abrupt fall or movement of a subject and provides an alert to the remote monitoring station (32) to indicate a probable medical emergency condition.

1. A medical monitoring system (100) that enables remote monitoring of at least one physiological parameter of a human subject comprising:
at least one wearable monitoring device (10) that serves to monitor at least one physiological parameter of a subject that includes but is not limited to body temperature, perspiration or blood pressure by acquiring at least one electrically embodied physiological parameter measurement corresponding to the at least one physiological parameter of the subject;
a wireless transceiver unit (34), that is configured to communicate with the wearable monitoring device (10) via wireless communication;
wherein, the wireless transceiver unit (34) is further configured to communicate a medical alert signal and the at least one electrically embodied physiological parameter measurement communicated from the wearable monitoring device, to a remote monitoring station (32) via any one or a combination of wireless communication, wire-line communication and/or optical communication.

2. The medical monitoring system according to claim 1, wherein the wearable monitoring device (10) acquires the at least one electrically embodied physiological parameter measurement by way of periodically sampling of the at least one electrically embodied physiological parameter measurement of the subject.

3. The medical monitoring system according to claim 1, wherein the wearable monitoring device (10) continuously acquires the at least one electrically embodied physiological parameter measurement corresponding to the at least one electrically embodied physiological parameter measurement of the subject.

4. The medical monitoring system (100) according to claim 1, wherein the wireless communication between the wearable monitoring device (10) and the wireless transceiver unit (34) is conducted using a communication module selected from the group consisting of IEEE's Bluetooth Protocol, IEEE's Zig-Bee Protocol, IEEE's 802.11 Protocol and RF signaling.

5. The medical monitoring system (100) according to claim 1, wherein the wearable monitoring device (10) compares the at least one electrically embodied physiological parameter measurement with threshold values of the at least one electrically embodied physiological parameter measurement pre-stored in a memory module (15b) and produces a medical alert signal in the event the at least one electrically embodied physiological parameter measurement falls beyond predetermined threshold values.

6. The medical monitoring system (100) according to claim 1, wherein the at least one wearable monitoring device (10) transmits the medical alert signal and the at least one electrically embodied physiological parameter measurement by embodying said medical alert signal and said at least one electrically embodied physiological parameter measurement as RF signals to the wireless transceiver unit (34).

7. The medical monitoring system (100) according to claim 1, wherein remote monitoring station (32) stores said electrically embodied physiological parameter measurements corresponding to the at least one physiological parameter of the human subject to perform historical trending.

8. A wearable monitoring device (10) for used in a medical monitoring system (100) as claimed in claim 1, wherein said wearable monitoring device (10) includes a memory module (15b) for housing a software application executable by a processing module (15f) to consequently enable the wearable monitoring device (10) to execute a plurality of actions that include one or more of the following actions:
i.) pairing of the least one wearable monitoring device (10) to the wireless transceiver unit (34) in cooperation with a software application housed within the memory module (34i) and executed by the processing module (34f);
ii.) acquiring at least one electrically embodied physiological parameter measurement from at least one physiological sensor (15a);
iii.) setting of customizable threshold settings of at least one physiological parameter measurement value stored within the memory module (15b);
iv.) comparing of at least one electrically embodied physiological parameter measurement obtained via the at least one physiological sensor (15a) with a threshold physiological parameter measurement value stored within the wearable monitoring device memory module (15b);
v.) actuating a local alert, in the event the at least one electrically embodied physiological parameter measurement falls beyond the threshold physiological parameter measurement value, and/or actuating a transmission of a medical alert signal to the wireless transceiver unit (34) via a RF transceiver circuit (15g) and a RF antenna (15h); and
vi.) actuating the transmission of at least one electrically embodied physiological parameter measurement to the wireless transceiver unit via the RF transceiver circuit (15g) and the RF antenna (15h).

9. The wearable monitoring device (10) as claimed in claim 8, wherein said wearable monitoring device (10) includes electronics which includes any one of or a combination of at least one display unit (12), an electrical power source (15d), an electrical power source charging port (20) for charging the electrical power source (15d), and a manual emergency alert activation button (22).

10. The wearable monitoring device (10) as claimed in claim 8, wherein said wearable monitoring device (10) includes electronics which includes any one of or a combination of at least one electrical power source charge management circuit (15c) and a power management circuit (15e).

11. The wearable monitoring device (10) as claimed in claim 8, wherein said physiological sensor (15a) includes any one of or a combination of a temperature sensing element and relative humidity sensing element.

12. The wearable monitoring device (10) as claimed in claim 8, wherein said physiological sensor (15a) includes at least one sensing element that is a capacitive sensing element that does not contact with a surface of skin of a subject to provide a measure of the subject's perspiration in terms of relative humidity.

13. A wireless transceiver unit (34) for use in a medical monitoring system (100) as claimed in claim 1, wherein the wireless transceiver unit (34) includes a memory module (34i) for housing a software application executable by a processing module (34f) to consequently enable the wireless transceiver unit (34) to execute a plurality of actions that include one or more of the following actions:
i.) pairing (i.e. performs a handshaking procedure to establish a bi-directional communication channel between at least one wearable monitoring device (10) and a wireless transceiver unit (34) of the least one wearable monitoring device to the wireless transceiver unit (34);
ii.) setting of customizable threshold settings of at least one physiological parameter measurement value stored within the memory module (34i);
iii.) acquiring a wirelessly transmitted RF signal embodying a medical alert signal and/or a RF signal embodying at least one electrically embodied physiological parameter measurement via the first RF antenna (34h) and a first RF transceiver circuitry (34g);
iv.) comparing of a received at least one electrically embodied physiological parameter measurement with a threshold physiological parameter measurement value at the processing module (34f);
v.) actuation of a local alert at the wearable monitoring device and/or at the wireless transceiver unit and/or a transmission of a medical alert signal to a remote monitoring station via any one of or a combination of any one of a wireless communication channel, a fiber-optic communication channel or a wire-line communication channel; and
vi.) actuating a transmission of a received at least one electrically embodied physiological parameter measurement to a remote monitoring station (32) via any one of or a combination of any one of a wireless communication channel, a fiber optic communication channel or a wire-line communication channel.

14. The wireless transceiver unit (34) as claimed in claim 13, wherein the wireless transceiver unit includes a first RF antenna (34h), a first RF transceiver circuit (34g), a second RF transceiver circuit (34c), a second RF antenna (34d), a display module (34a), and an alphanumeric keypad module (34j).

15. The wireless transceiver unit (34) as claimed in claim 13, wherein said threshold settings are customizable to accommodate an age, gender, normal medical condition of a subject being monitored and an amount and type of activities that a subject may undertake during particular time periods within a day.

16. The wireless transceiver unit (34) as claimed in claim 13, wherein the medical alert signal transmitted to a remote monitoring station (32) includes an SMS alert message embodied as an RF signal and a call signal embodied as an RF signal.

17. A method for use in a medical monitoring system (100) comprising of at least one wearable monitoring device (10) and a wireless transceiver unit (34) to determine and alert a subject wearing the at least one wearable monitoring device, as to whether, the wearable monitoring device (10) is in firm contact with the subject's skin utilizing an electrically embodied physiological parameter measurement obtained by an at least one physiological sensor (15a) of the at least one wearable monitoring device (10); the method being embodied as a software application residing within a wearable monitoring device memory module (15b) of the wearable monitoring device (10) and/or a wireless transceiver unit memory module (34i) of the wireless transceiver unit (34) and comprises the steps of:
i.) acquiring an electrically embodied physiological parameter measurement;
ii.) determining if the electrically embodied physiological parameter measurement acquired in step (i) falls beyond a predetermined range of measurements that represent the normal range of measurement values when the wearable monitoring device (10) is tightly coupled to the subject's skin; and
iii.) actuating an alert in the event said electrically embodied physiological parameter measurement does not fall within a normal range of measurement values.

18. The method according to claim 17, wherein the electrically embodied physiological parameter measurement is a body temperature measurement.

19. A method to alert a remote care-giver or a remote team of emergency response personnel via a remote monitoring station (32) through a wireless transceiver unit (34) that is in communication with a wearable monitoring device (10) and said remote monitoring station (32); the method being embodied as a software application residing within a wireless transceiver unit memory module (34i) of the wireless transceiver unit (34) and comprises the steps of:
i.) acquiring at least one electrically embodied physiological parameter measurement from a wearable monitoring device (10) worn by a subject who is being monitored;
ii.) determining if the at least one electrically embodied physiological parameter measurement acquired in step (i) falls beyond a predetermined range of measurements that represent the normal range of physiological parameter measurement values when the subject is not experiencing a medical emergency condition;
iii.) actuating a local alarm on the wearable monitoring device (10);
iv.) automatically executing a phone call to at least one predetermined primary number corresponding to at least one remote monitoring station (32); the phone call being executed a predetermined number of times until it is acknowledged by the remote monitoring station (32);
v.) automatically executing a phone call to at least one alternate predetermined secondary number corresponding to at least one remote monitoring station (32) a predetermined number of times until it is acknowledged, in the event the phone call to the at least one predetermined primary number is not acknowledged;
vi.) repeating steps (iv) and (v) until either one of the phone calls to the at least one predetermined primary number or the at least one predetermined secondary number is acknowledged; and
vii.) automatically executing the wireless transmission of an SMS alert to the at least one predetermined primary and/or secondary number corresponding to at least one remote monitoring station (32).

20. A method according to claim 19, wherein the remote monitoring station (32) is selected from the group consisting of a cellular phone of a remote care-giver and a call-center associated with a medical emergency response team.
